(19) United States
(12) Patent Application Publication
(10) Pub. No.: US 2022/0401621 A1
(43) Pub. Date: Dec. 22, 2022

(54) Preservation Of Neuron Health And Regenerative Capacity Following Nervous System Injury

(71) Applicants: THE TRUSTEES OF THE UNIVERSITY OF PENNSYLVANIA, Philadelphia, PA (US); The United States of America as represented by the Department of Veterans Affairs, Washington, DC (US)

(72) Inventors: Daniel Kacy Cullen, Media, PA (US); Justin C. Burrell, Philadelphia, PA (US); Joseph Cole Maggiore, Pittsburgh, PA (US)

(21) Appl. No.: 17/776,829

(22) PCT Filed: Nov. 18, 2020
(86) PCT No.: PCT/US2020/061087

§ 371 Date: May 13, 2022

Related U.S. Application Data
(60) Provisional application No. 62/936,983, filed on Nov. 18, 2019.
(60) Provisional application No. 62/937,489, filed on Nov. 19, 2019.

(51) Int. Cl. A61L 27/36; A61L 27/38

(52) U.S. Cl. CPC A61L 27/3675; A61L 27/383; A61L 27/3834; A61L 27/3633; A61L 27/3878; A61L 2300/62; A61L 2430/32

(57) ABSTRACT

In various aspects and embodiments, the present invention provides methods for maintaining motor neuron health in the spinal cord and pro-regenerative capacity of a proximal nerve segment subsequent to a nerve injury in a subject in need thereof, the methods comprising transplanting a stretch-grown tissue engineered nerve graft (TENG) into a proximal site contacting the proximal nerve segment.

### CROSS-REFERENCE TO RELATED APPLICATION

This application claims priority to U.S. Provisional Application Ser. No. 62/936,983 filed Nov. 18, 2019 and U.S. Prov. Appl. No. 62/937,489 filed Nov. 19, 2019, the content of each of which is incorporated by reference herein in its entirety.

### STATEMENT REGARDING FEDERALLY SPONSORED RESEARCH OR DEVELOPMENT

This invention was made with government support under grant number 101-BX003748 awarded by the Department of Veterans Affairs and grant numbers W81XWH-16-1-0796 and W81XWH-15-1-0466 awarded by the Department of Defense. The government has certain rights in the invention.

### BACKGROUND OF THE INVENTION

It is estimated that nearly 20 million Americans suffer from traumatic peripheral nerve injury (PNI) and only 50% of patients achieve normal functional recovery following surgery. Following PNI, the distal nerve segment undergoes Wallerian degeneration—a process of rapid axonal loss and myelin sheath degradation. During this process, contrary to degeneration, Schwann cells proliferate—forming highly aligned columns, called Bands of Büngner. These facilitate regeneration and maintain neuronal cell health proximally by providing a pro-regenerative environment that counteracts the cell death process induced from PNI trauma. However, the pro-regenerative environment cannot be sustained in cases requiring long regenerative distances to the distal muscle and/or organ end-targets. Moreover, prolonged denervation results in permanent muscle atrophy, diminished proximal neuronal health, retrograde dieback of axotomized neurons, and ultimately an overall lowering of regenerative capacity for functional recovery. For this reason, a PNI repair strategy that provides a pro-regenerative environment—including maintenance of proximal neuronal cell health—is necessary to establish a more comprehensive and effective clinical repair strategy.

In particular, one important cellular process involved with maintaining neuronal cell health is retrograde transport. Studies have shown that loss of retrograde transport in proximal neuron cell bodies reduces neuronal health during regeneration. Following untreated PNI, retrograde transport of neurotrophic factors is inhibited in the proximal stump, concurrent with an initiation of a retrograde dieback process. Over time, sustained diminished retrograde transport leads to poor neuronal cell health, and regenerative capacity of proximal neurons in the spinal cord. Thus, retrograde transport is a valuable surrogate marker for neuronal cell health and ultimately regenerative capacity. Recent studies have demonstrated that neurotrophic signals such as glial cell-line derived neurotrophic factor (GDNF), brain derived neurotrophic factor (BDNF) and nerve growth factor (NGF) secreted in the distal environment revitalize retrograde transport, improve cell health, and sustain regeneration. Recently, biological nerve grafts have been developed containing exogenous growth factors to simulate a pro-regenerative environment. However, a bolus of exogenous growth factors is unlikely able to provide a sustainable pro-regenerative environment required for functional recovery. Despite the recent advancements in neural engineering, autograft nerve repairs still remain the current gold standard surgical treatment due to the superior functional recovery compared to commercially available products, such as a nerve guidance tube (NGT) or acellular nerve allograft. As an endogenously-available living scaffold, autografts provide regenerating host axons with a structural support, necessary for anisotropic growth, as well as a rich supply of growth factors secreted by the Schwann cells. However, autografts pose a challenging repair solution for instances of large gap repair and donor site comorbidity.

There is a need in the art for improved methods of nerve repair. This disclosure addresses that need.

### SUMMARY OF THE INVENTION

In one aspect, the disclosure provides a method for maintaining the health of motor neurons in the spinal cord and the pro-regenerative capacity of a proximal nerve segment subsequent to a nerve injury in a subject in need thereof, the method comprising transplanting a stretch-grown tissue engineered nerve graft (TENG) into a proximal site contacting the proximal nerve segment.

In various embodiments, the subject is a mammal.

In various embodiments, the subject is a human.

In various embodiments, the nerve injury comprises an injury to a peripheral or cranial nerve of a subject.

In various embodiments, the nerve injury comprises an injury to the spinal cord of a subject.

In various embodiments, the nerve injury comprises the loss of a segment of nerve.

In various embodiments, the nerve injury comprises a nerve lesion of less than about 1 cm in length.

In various embodiments, the nerve injury comprises a nerve lesion of at least about 1 cm in length.

In various embodiments, the nerve injury comprises a nerve lesion of at least about 3 cm in length.

In various embodiments, the nerve injury comprises a nerve lesion of at least 5 cm in length.

In various embodiments, the nerve injury comprises multiple nerve lesions.

In various embodiments, the pro-regenerative capacity of the proximal nerve segment is maintained for at least about 16 weeks.

In various embodiments, the pro-regenerative capacity of the proximal nerve segment is maintained until at least such time as proximal nerve axons regenerate across the nerve injury.

In various embodiments, pro-regenerative capacity of the proximal nerve segment is maintained until at least such time as proximal nerve axons reinnervate distal targets.

In various embodiments, the method further comprises a primary procedure for nerve repair.

In various embodiments, the method results in a greater degree of functional recovery following repair of PNI, as compared to the degree of functional recovery that occurs following the primary procedure alone.

In various embodiments, the method is conducted in the absence of any other nerve repair.

In various embodiments, the method further comprises providing a neurotrophic factor, culture supernatant, or cells to the proximal nerve segment.

In various embodiments, wherein the cells are selected from the group consisting of neurons, and stem cells.

In various embodiments, the method does not comprise transecting a nearby healthy nerve or the repaired nerve.

In various embodiments, the method enhances the survival of Schwann cells in the proximal nerve segment.

In various embodiments, the proximal site is at least about 3 cm away from the site of injury.

In various embodiments, the proximal site is less than least about 3 cm away from the site of injury.

In various embodiments, the method comprises contacting multiple proximal nerve segments with one or more stretch-grown TENG.

In various embodiments, the TENG facilitates axon growth and Schwann cell infiltration, thereby maintaining the pro-regenerative capacity of the proximal nerve segment.

In various embodiments, the method further comprises transplanting a stretch-grown tissue engineered nerve graft (TENG) into a distal site in the distal nerve segment.

In various embodiments, the TENG is a forced aggregation TENG.

In various embodiments, the TENG is transplanted into a proximal site in a peripheral nerve, a cranial nerve or a spinal nerve of a subject.

In another aspect, the disclosure provides a method for maintaining the health of motor neurons in the spinal cord and the pro-regenerative capacity of a proximal nerve segment subsequent to a nerve injury by transplanting one or more neurons into a proximal site in the proximal nerve segment, wherein the one or more neurons facilitate regeneration and functional following nerve repair.

In various embodiments, the nerve injury comprises an injury to a peripheral nerve, a cranial nerve or a spinal cord nerve of the subject.

In various embodiments, one or more neurons are transplanted into a peripheral nerve, a cranial nerve or a spinal cord nerve of the subject.

In various embodiments, one or more neurons are injected into a proximal nerve stump of the subject.

In various embodiments, one or more neurons are transplanted in a delivery device that is secured to the proximal nerve stump.

In various embodiments, one or more neurons are encapsulated in extracellular matrix sheath and transferred into the delivery device.

In various embodiments, one or more neurons are stretch-grown in culture, encapsulated in extracellular matrix, and transferred into the delivery device.

In various embodiments, one or more neurons are pre-encapsulated in extracellular matrix, grown in culture, and then transferred into the delivery device.

In various embodiments, one or more neurons are comprise glutamatergic, GABAergic, sensory neurons, motor neurons or combinations thereof.

In various embodiments, one or more neurons are accompanied by Schwann cells, macrophages, fibroblasts, mesenchymal stem cells or myocytes.

In various embodiments, one or more neurons is derived from neuronal progenitor cells.

In various embodiments, one or more neurons is derived from stem cells.

In various embodiments, one or more neurons is derived from embryonic cells.

In various embodiments, the one or more neurons are implanted along or within an extracellular matrix core and are formed via forced cell aggregation.

In various embodiments, one or more neurons, wherein one or more neurons are transduced to overexpress neurotrophic factors, including, but not limited to GDNF, BDNF, VEGF, or NGF.

In various embodiments, one or more neurons are supplemented with cells transduced to overexpress neurotrophic factors, including, but not limited to GDNF, BDNF, VEGF, or NGF.

In various embodiments, the nerve injury comprises the loss of a segment of nerve.

In various embodiments, the nerve injury comprises a nerve lesion of less than about 1 cm in length.

In various embodiments, the nerve injury comprises a nerve lesion of at least 1 cm in length.

In various embodiments, the nerve injury comprises a nerve lesion of at least about 3 cm in length.

In various embodiments, the nerve injury comprises a nerve lesion of at least about 5 cm in length.

In various embodiments, the nerve injury comprises multiple nerve lesions.

In various embodiments, the pro-regenerative capacity of the proximal nerve segment is maintained for at least about 16 weeks.

In various embodiments, the pro-regenerative capacity of the proximal nerve segment is maintained until at least such time as proximal nerve axons regenerate across the nerve injury.

In various embodiments, pro-regenerative capacity of the proximal nerve segment is maintained until at least such time as proximal nerve axons reinnervate distal targets

In various embodiments, the method further comprises a primary procedure for nerve repair that results in a greater degree of functional recovery following repair of PNI, as compared to the degree of functional recovery that occurs following the primary procedure alone.

In various embodiments, the method further comprises a primary procedure to preserve the regenerative capacity of the proximal nerve segment followed by a delayed nerve repair at a later time point that results in a greater degree of functional recovery following repair of PNI, as compared to the degree of functional recovery that occurs following the delayed repair alone.

In various embodiments, the method is conducted in the absence of any other nerve repair.

In various embodiments, the nerve injury occurs as a result of a trauma.

In various embodiments, wherein the nerve injury occurs as a result of a surgical procedure.

In various embodiments, the nerve injury occurs as a result of patient positioning during surgery.

In various embodiments, the nerve injury occurs as a result of a compression injury or a crush injury.

In various embodiments, the injury occurs as a result of a disease or condition relating to a loss of motor or sensory nerve function.

In various embodiments, the injury occurs as a result of a congenital anomaly.

In various embodiments, the injury occurs as a result of an amputation.

In various embodiments, the injury occurs as a result of complete or partial removal of an organ, tumor or tissue.

In various embodiments, the injury occurs as a result of a metabolic/endocrine complication, inflammatory disease, autoimmune disease, vitamin deficiency, infectious disease, toxin, exposure to organic metal or heavy metal, or administration of a medication or drug.

In various embodiments, the method further comprises providing a neurotrophic factor, culture supernatant, or cells to the distal nerve segment.

In various embodiments, the method does not comprise transecting a nearby healthy nerve or the repaired nerve.

In various embodiments, the method does not comprise transecting a nearby healthy nerve or the repaired nerve.

In various embodiments, the distal site is less than least about 3 cm away from the site of injury.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1C: Biological Implications of Living and Non-Living Repair Strategies for Segmental Nerve Defects and Example of Axonal “Stretch-Growth” for Bioengineered Living Scaffold. FIG. 1A: After segmental nerve injury, successful functional recovery necessitates that axons regenerate across a scaffold bridging the proximal and distal nerve stumps, enter the denervated distal nerve, and extend the entire distance to the end target. Often overlooked is that the surgical repair strategy may directly impact the ceiling for functional recovery. Autografts are natural living scaffolds that provide pro-regenerative trophic support, and chemotaxic and haptotaxic cues that enable accelerated axonal regeneration across the graft and is anticipated to preserve spinal motor neuron health, which would allow for greater functional recovery. Conversely, acellular strategies often lack neurotrophic and/or chemotaxic cues, leading to disorganized axonal growth, poor neuronal health, and ultimately diminished regenerative capacity. The present disclosure encompasses recently developed tissue engineered nerve grafts (TENGs) comprised of stretch grown axons that are bioengineered living scaffolds that preserve the ceiling for functional recovery by providing trophic support for regenerating axons, which is expected to also preserve the regenerative capacity of the proximal neurons. (FIGS. 1B-1C) To generate TENGs, embryonic rat GFP⁺ DRG explants were plated as two populations across a towing membrane separated by 500 μm. For 5 days in vitro, the DRG axons integrated and then were stretched to 1 cm with a micro-stepper over 6 days in a custom mechanobioreactor for transplantation. Example images are shown in FIG. 1B phase contrast and FIG. 1C and fluorescent microscopy. Once the axons reach the desired length, the TENG is encapsulated in collagen and rolled prior to transplantation. Scale bars: (Macro) 1000 μm; (Zoom in) 100 μm.

FIGS. 2A-2D: Host Axon Regeneration and Schwann Cell Infiltration at 2 Weeks Following 1 cm Lesion in Rat Sciatic Nerve. Confocal reconstruction of longitudinal frozen sections (20 μm) of a 1 cm rodent sciatic nerve segmental repair using FIG. 2A: autograft, FIG. 2B: NGT, FIG. 2C: NGT+DRG, and FIG. 2D: TENG. (a-d) Higher magnification images showing sections labeled for regenerating axons (SMI31/32⁺) and Schwann cells (S100). Transplanted DRG neurons expressing GFP were observed in the repair zone of the (FIG. 2C) NGT-DRG and (FIG. 2D) TENG groups. FIGS. 2A and 2D: Robust host axonal ingrowth and Schwann cell infiltration was observed across the autograft and TENG. Comparatively, axon extension and Schwann cell infiltration were attenuated following repair with NGT (FIG. 2B). FIGS. 2C and 2D: Transplanted DRGs survived and facilitated host axon across the graft. (d, d″) Higher magnification images revealed TENG neuronal survival post transplantation and close integration with host Schwann cells and axons. (d′) Host axons extending across the repair zone were often found near TENG axons spanning the length of graft. (d′″) TENG and host axons were also observed extending into the distal nerve. Scale: A-C 1000 μm; a-c 100 μm.

FIGS. 3A-3G: FR and NeuN Quantification in Naïve Spinal Cord Tissue to Validate Optical Clearing Technique with Conventional IHC Methodology. Visikol HISTO protocol can be used to analyze spinal cord tissue longitudinally or axially in 500 μm thick macro sections. FIG. 3A: Fluoro-Ruby labelled cells and NeuN immunostained cells can be visualized in maximum projection z-stacks. Scale bars: 3000 μm, 600 μm, 60 μm. FIG. 3B: NeuN antibody penetration was visually confirmed through the complete 3D confocal reconstruction. XYZ coordinate plane three-dimensional view of axial spinal cord ventral horn section labelled with Fluoro-Ruby and NeuN. FIG. 3C: XY 3D view of axial spinal cord ventral horn section. FIG. 3D: XZ view of axial spinal cord section, confirming sufficient laser penetration to view Fluoro-Ruby through entirety of section. FIG. 3E: Standard IHC (30 μm histological samples), z-stack maximum projection. FIG. 3F: Spinal cord tissue blocks (500 μm sections) stained for NeuN IHC during Visikol HISTO process, z-stack maximum projection. Scale bar: 300 μm. FIG. 3G: No statistical significance was found between 30 μm sections stained using conventional IHC with Abercrombie correction (IHC+AC) and Visikol cleared tissue. Error bars represent standard deviation. ***=p<0.001; ****=p<0.0001.

FIGS. 4A-4H: FR Retrograde Tracing and motor neuron (MN) Quantification in the Ventral Horn Spinal Cord. FIGS. 4A-4E: MN cell bodies labeled with FR and NeuN were visualized in the ventral horn of optically cleared spinal cords. FIG. 4F: FR⁺ MN cell bodies in the ventral horn were quantified. When compared with naïve, both NGT (p<0.0001) and NGT+DRG (p<0.05) repair strategies produced a statistically significant decrease in mean count. FIG. 4G: NeuN cell bodies were quantified in the ventral horn and a statistically significant reduction was observed following the NGT repair group compared to naïve (p<0.05). Scale bar: (Macro) 150 μm; (Zoom in) 150 μm. Error bars represent SEM. ns denotes no significance compared to naïve. FIG. 4H: Frequency distributions of MN FR fluorescence intensity were plotted. Fluorescence intensity was calculated for each FR⁺ cell in the ventral horn by measuring the maximum intensity of the cell relative to the local background. Individual cell intensity was log transformed to fit a normal distribution. See FIGS. 9A-9E for frequency distribution profiles for each experimental group relative to the naïve frequency distribution.

FIGS. 5A-5H: FR Quantification in L4 and L5 DRG. L4/L5 DRG samples were visualized en bloc following whole mount optical tissue clearance. FIGS. 5A-5F: Representative confocal reconstructions generated from z-stack max projections are shown. FIG. 5G: No statistical difference was observed between any treatment group for L4 and L5 DRG FR counts. Error bars represent SEM. Scale bar: (FIG. 5A) 700 μm; (FIGS. 5B-5F) 50 μm. FIG. 5H: Frequency distributions of DRG fluorescence intensity were plotted. Fluorescence intensity was calculated for each FR⁺ cell in the ventral horn by measuring the maximum intensity of the cell relative to the local background. Individual cell intensity was log transformed to fit a normal distribution. See FIGS. 10A-10E for frequency distribution profiles for each experimental group relative to the naïve frequency distribution.

FIGS. 6A-6C: FR Uptake in Spinal Motor Neurons and DRGs at 16 Weeks Post Repair. In a subset of animals, spinal motor neurons (FIG. 6A) and DRGs (FIG. 6B) were labeled with FR at 16 weeks following peripheral nerve repair. Qualitative assessment revealed similar number of labeled spinal motor neurons following TENG and autograft repair and no differences were observed in the DRG between groups. FIG. 6C: Similar muscle electrophysiological recovery was observed between the TENG and AG repair groups. Scale bar: 100 μm.

FIG. 7: Schematic of Fluoro-Ruby (FR) Application and Retrograde Transport. This figure illustrates a process wherein a retrograde fluorescent dye (FR) was applied at 2 weeks following repair of a 1 cm segmental nerve repair in a rat model. FR was applied proximal to the graft site, and the nerve graft zone (distal) was harvested for histological analysis. At 3 days post FR application, the animal was euthanized and the spinal cord and DRG were harvested for histological analyses.

FIG. 8: Spinal Cord Tissue Clearing, Staining, and Imaging Workflow Schematic. The surgical site was re-exposed at the terminal time and the graft was removed for further histological analysis. Fluororuby was applied to the proximal nerve stump and the animal was returned to the colony for an additional three days. Following transcardial perfusion, the spinal cord was harvested and blocked for optical clearing. After identifying the labeled tissue blocks, a subset were stained, optically cleared, and imaged using confocal microscopy. Positively labeled cells were then quantified from z-stack max projections. A subset of optically-cleared samples were reverse cleared for validation compared to traditional sectioning and quantification.

FIGS. 9A-9E: FR Intensity in Ventral Horn Motor Neuron Population. FIG. 9A: Frequency distributions of MN fluorescence intensity were plotted. Intensity of FR fluorescence was calculated for each FR⁺ cell using maximum intensity of the cell relative to the local background around the cell. Individual cell intensity was log transformed to fit a normal distribution. FIGS. 9B-9E: Frequency distributions for each experimental group were compared to the naïve frequency distribution. Note (A) is reproduced from FIG. 4H for convenience.

FIGS. 10A-10E: FR Intensity in L4 DRG. FIG. 10A: Frequency distributions of DRG fluorescence intensity were plotted. Intensity of FR fluorescence was calculated for each FR⁺ cell using maximum intensity of the cell relative to the local background around the cell. Individual cell intensity was log transformed to fit a normal distribution. FIGS. 10B-10E: Frequency distributions for each experimental group were compared to the naïve frequency distribution. Note FIG. 10A is reproduced from FIG. 5H for convenience.

FIGS. 11A-11D: Various TENGS that may be useful in certain embodiments of the invention. FIG. 11A: Neurons encapsulated in extracellular matrix (ECM). FIG. 11B: Stretch-grown neurons and axons encapsulated in ECM. FIG. 11C: Neurons pre-encapsulated in ECM encased within a hydrogel biomaterial. FIG. 11D: Neurons+axons pre-encapsulated in ECM encased within a hydrogel biomaterial.

FIG. 12A: TENG nerve repair for all peripheral nerve injuries of any deficit length and at any position. A primary nerve repair with a TENG provides simultaneous maintenance of the spinal cord motor neurons and sensory neuron DRGs, while also allowing for sustained regenerative capacity and maintenance of the pro-regenerative distal nerve Schwann cells and muscle and/or organ end-targets for all deficit lengths and locations, such as long gap nerve injury (>3 cm), short gap nerve injury (<3 cm), and proximal and/or distal nerve injury.

FIG. 12B: TENG transplantations as a babysitting repair strategy at any position Primary proximal nerve repair with distal satellite graft to “babysit” distal pathway and end-target muscle: Following a primary nerve repair with any reconstructive strategy, such as an autograft, tube, or TENG, a distal satellite graft (“distal babysitter TENG”) can be transplanted either end-to-side or in-continuity with the nerve or injected directly into the fascicle. Distal babysitter TENGs maintain the pro-regenerative distal nerve Schwann cells and the muscle and/or organ end-targets (“Application B 1”). In a clinical scenario, sometimes nerve reconstruction is not immediately feasible. Here, primary nerve repair with any reconstructive strategy, such as an autograft, tube, or TENG, a proximal satellite graft (“proximal babysitter TENG”) can be transplanted either end-to-side or in-continuity with the nerve or injected directly into the fascicle. Proximal babysitter TENGs maintain spinal cord motor neurons and sensory neuron DRGs, allowing for sustained regenerative capacity (“Application B2”).

FIG. 12C: TENG living scaffolds to “babysit” the proximal spinal cord motor neurons and sensory DRG neurons, as well as the pro-regenerative environment in the distal nerve pathway. Proximal and distal “babysitting” TENGs improves outcome following delayed repair: Proximal babysitter TENGs maintain spinal cord motor neurons and sensory DRG neurons, allowing for sustained regenerative capacity (“Application C1”). Distal babysitter TENGs maintain the pro-regenerative distal nerve Schwann cells and the muscle and/or organ end-targets (“Application C2”). Babysitter grafts maintain the proximal spinal cord and DRG neuronal regenerative capacity and the pro-regenerative environment in the distal nerve, which would enhance regeneration and functional recovery following delayed nerve repair using any reconstructive strategy, such as an autograft, tube, or TENG at any position (“Application C3”)

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting. As used herein, the singular form “a,” “an,” and “the” include plural references unless the context clearly dictates otherwise.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

“Isolating,” means to obtain one or more types of cells, purify to remove or substantially remove other cells types and grow in primary culture.

“Proximal nerve stump” as used herein means the nerve stump proximal to the injury site.

A “subject” or “patient,” as used therein, may be a human or non-human mammal. Non-human mammals include, for example, livestock and pets, such as ovine, bovine, porcine, canine, feline and murine mammals. Preferably, the subject is human.

“Tissue engineered axonal tracts” refer to living axonal tracts generated from TENGs, in which the neuronal cell bodies have been severed leaving only axonal tracts. In various embodiments the TENG may have been generated from any sub-type of neuron, including but not limited to neurons from the peripheral nervous system (e.g., spinal motor, sensory dorsal root ganglia), central nervous system (e.g., glutamatergic, GABAergic, dopaminergic, serotonergic), and autonomic nervous system (e.g, ganglionic norepinephrinergic, acetycholinergic, or dopaminergic).

“Tissue-Engineered Nerve Grafts (TENGs)” is used interchangeably herein with the term “stretch-grown TENG” and refers to living three-dimensional nerve constructs that consist of neurons, including neuronal cell bodies, and longitudinally aligned axonal tracts.

“Forced aggregation TENG” and “forced cell aggregation TENG” are used interchangeably to refer to a TENG that is stretch grown from an aggregate or sphere of neurons formed by forced aggregation.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Without meaning to be limited by theory, in one aspect the invention is based in part on the unexpected discovery that subsequent to a nerve injury subjects benefit from maintenance of pro-regenerative capacity of the proximal nerve segment. Accordingly, in one aspect the invention provides a method for maintaining the health of motor neurons in the spinal cord and the pro-regenerative capacity of a proximal nerve segment subsequent to a nerve injury in a subject in need thereof, the method comprising transplanting a stretch-grown tissue engineered nerve graft (TENG) into a proximal site contacting the proximal nerve segment. In various embodiments, the TENG may be a forced aggregation TENG.

The method is widely applicable in various subjects and with regard to various injuries. In various embodiments, the subject is a mammal. In various embodiments, the subject is a human. In various embodiments, the nerve injury comprises an injury to a peripheral nerve of a subject. In various embodiments, the nerve injury comprises an injury to the spinal cord of a subject. In various embodiments, the nerve injury includes a crush injury without the loss of a segment of nerve. In various embodiments, the nerve injury comprises the loss of a segment of nerve. In various embodiments, the nerve injury comprises a nerve lesion of less than about 1 cm in length (e.g., 0.9 cm, 0.8 cm. 0.7 cm, 0.6 cm. 0.5 cm, 0.4 cm, 0.3 cm, 0.2 cm 0.1 cm, or less). In various embodiments, the nerve injury comprises a nerve lesion of at least about 1 cm in length. In various embodiments, the nerve injury comprises a nerve lesion of at least about 3 cm in length (e.g., 3.5 cm, 4 cm, 4.5 cm, 5 cm or greater). In various embodiments, the nerve injury comprises a nerve lesion of at least 5 cm in length (e.g., 6 cm, 7 cm, 8 cm, 9 cm, 10 cm or greater). In various embodiments, the nerve injury comprises multiple nerve lesions. In various embodiments, the pro-regenerative capacity of the proximal nerve segment is maintained for at least about 16 weeks (e.g., 17 weeks, 18 weeks, 19 weeks, 20 weeks or more). In various embodiments, the pro-regenerative capacity of the proximal nerve segment is maintained until at least such time as proximal nerve axons regenerate across the nerve injury. In various embodiments, a primary nerve repair procedure is performed at the time the TENG is implanted and the implanted TENG maintains the pro-regenerative capacity of the proximal nerve segment until at least such time as proximal nerve axons reinnervate distal targets.

In various embodiments, the method further comprises a primary procedure for nerve repair. A person of ordinary skill in the art will recognize that a wide variety of primary nerve repair techniques known in the art are suitable for use in combination with various embodiments of the invention. In various embodiments, the method results in a greater degree of functional recovery following repair of PNI, as compared to the degree of functional recovery that occurs following the primary procedure alone. In various embodiments, the method is conducted in the absence of any other nerve repair. In various embodiments, the method further comprises providing a neurotrophic factor, culture supernatant, or cells to the proximal nerve segment. In various embodiments, the cells are selected from the group consisting of neurons, and stem cells. In various embodiments, the method does not comprise transecting a nearby healthy nerve or the repaired nerve. In various embodiments, the method enhances the survival of Schwann cells in the proximal nerve segment. In various embodiments, the proximal site is at least about 3 cm away from the site of injury. In various embodiments, the proximal site is less than least about 3 cm away from the site of injury. In various embodiments, the method comprises contacting multiple proximal nerve segments with one or more stretch-grown TENG. In various embodiments, the TENG facilitates axon growth and Schwann cell infiltration, thereby maintaining the pro-regenerative capacity of the proximal nerve segment. In various embodiments, the method further comprises transplanting a stretch-grown tissue engineered nerve graft (TENG) into a distal site in the distal nerve segment.

In various embodiments, a TENG is injected into the proximal nerve stump, directly attached to the proximal nerve stump, or transferred into a secondary delivery device secured to the proximal nerve stump. In various embodiments the device is a nerve conduit, nerve wrap, or a nerve cap (i.e. sealed on one end with the open end in contact with the proximal stump).

Encompassed by the present disclosure is the recognition that the timing of nerve repair can vary based on, inter alia, the specific injury and/or provided composition employed in a particular application. For example, in various embodiments, primary nerve repair occurs immediately using neurons within a graft. In various embodiments, primary nerve repair occurs in a delayed setting, where neurons in the proximal nerve stump preserve the regenerative capacity and then the primary repair is completed at a later time point. In various embodiments, primary nerve repair occurs at least about one day, at least about three days, at least about one week, at least about two weeks or at least about one month, at least about three months, at least about six months, at least about one year, or at least about five years after the time of injury. In various embodiments, a TENG is excised and a nerve repair procedure is performed. In various embodiments, a graft is placed between the proximal and distal nerve stumps

In various embodiments a TENG may comprise glutamatergic neurons, GABAergic neurons, sensory neurons, and/or motor neurons. In various embodiments, neurons may be derived from stem cells, neuroprogenitor cells, primary embryonic neurons (xenogenic or human) and/or embryonic stem cells.

In various embodiments at least some neurons are engineered (e.g., virally transduced) to overexpress one or more neurotrophic factors. In various embodiments, the neurotrophic factors including glial cell-line derived neurotrophic factor (GDNF), brain derived neurotrophic factor (BDNF) and nerve growth factor (NGF) vascular endothelial growth factor (VEGF). In various embodiments, a viral vector comprising a neurotrophic factor is added to the media containing the cells during the fabrication process.

In various embodiments, neurons may be supplemented with Schwann cells, macrophages, fibroblasts, mesenchymal stem cells or myocytes to promote neuronal health. In various embodiments, at least some Schwann cells, macrophages, fibroblasts, mesenchymal stem cells or myocytes may be virally transduced to overexpress neurotrophic factors, including GDNF, BDNF, VEGF, NGF. Tissue Engineered Nerve Grafts (TENGs)

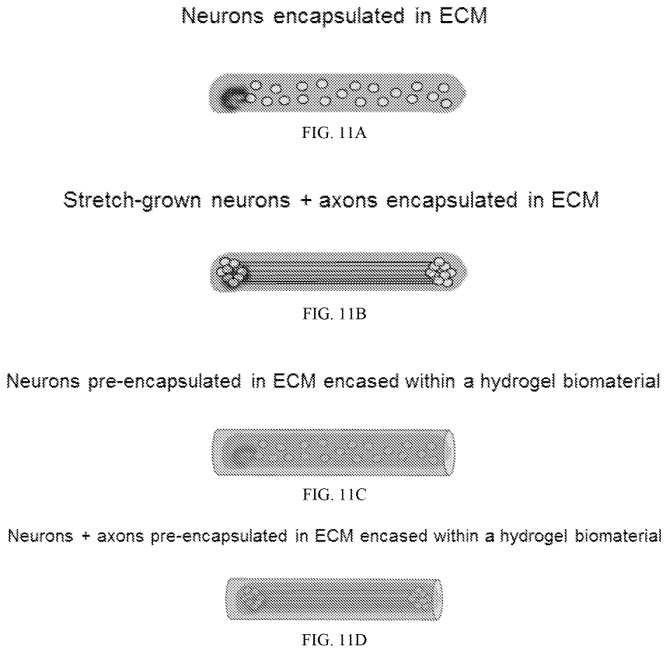

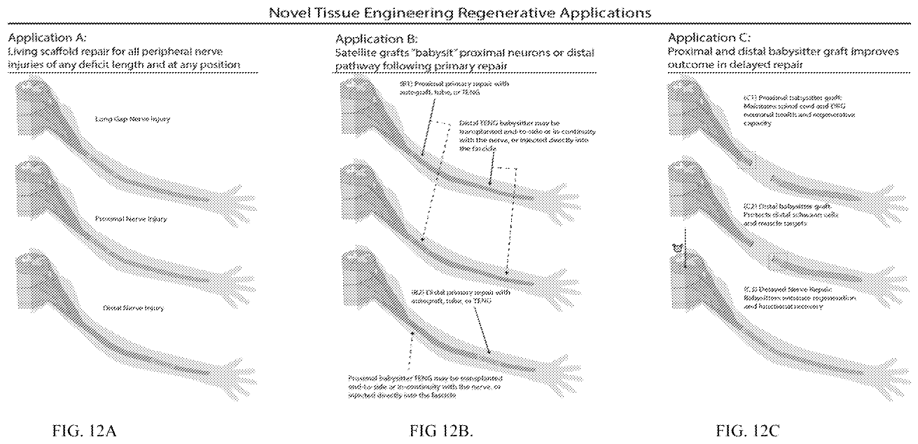

In various embodiments a TENG may be any TENG known in the art. TENGs that are suitable for use in various aspects and embodiments are described in U.S. Pat. No. 9,895,399, which is hereby incorporated by reference. Various TENGs that may be useful in certain embodiments are depicted in FIGS. 11A-11D. In various embodiments, a TENG comprises at least some neurons encapsulated in an extracellular matrix (ECM) sheath (FIG. 11A). In various embodiments, the neurons are encased in ECM and transplanted immediately.

In various embodiments, an ECM sheath comprises collagen, laminin, fibronectin, gelatin or a combination thereof. In various embodiments, a TENG comprises stretch-grown neurons and axons encapsulated in ECM (FIG. 11B). In various embodiments, neurons grown without ECM, ECM is added and transplanted immediately

In various embodiments, a TENG comprises neurons pre-encapsulated in ECM encased within a hydrogel biomaterial (FIG. 11C). In various embodiments, neurons (randomly organized) in ECM are grown in a hydrogel prior to transplantation.

In various embodiments, a hydrogel biomaterial comprises hyaluronan, chitosan, alginate, collagen, dextran, pectin, carrageenan, polylysine, gelatin, agarose, or a combination thereof. In various embodiments, a TENG comprises neurons and axons pre-encapsulated in ECM encased within a hydrogel biomaterial (FIG. 11D). In various embodiments, neurons separated by axons (i.e. stretch grown or populations of neurons placed on either end with axons grown to each other) encased in ECM grown in a hydrogel prior to transplantation.

In various embodiments, an extracellular matrix sheath has a first end and a second end. In various embodiments an extracellular matrix sheath is at least partially cylindrical with neurons implanted along at least a portion of the cylindrical extracellular matrix sheath. In various embodiments, neuronal bodies are placed at the first end of the extracellular matrix sheath and axons extend toward the second end along at least a portion of the extracellular matrix sheath. In various embodiments, neuronal bodies are located at the first and second end of the extracellular matrix sheath and axons extend along the extracellular matrix sheath from each population of neuronal bodies toward the opposite end through at least a portion of the extracellular matrix sheath.

In various embodiments, one or more neurons are implanted along or within an extracellular matrix core and may be formed via forced cell aggregation. In various embodiments, forced cell aggregation may be achieved by centrifuging a population of neuronal bodies in inverted pyramidal wells. In various embodiments, forced cell aggregation is achieved using the methods described in PCT Appl. No. PCT/US2017/027705, which is hereby incorporated by reference.

In another aspect, some embodiments provide methods for maintaining the health of motor neurons in the spinal cord and/or the pro-regenerative capacity of a proximal nerve segment subsequent to a nerve injury by transplanting one or more neurons into a proximal site in the proximal nerve segment, wherein the one or more neurons facilitate regeneration and/or functional recovery following nerve repair. In various embodiments, a nerve injury comprises an injury to a peripheral nerve, a cranial nerve and/or a spinal cord nerve of the subject. In various embodiments, a nerve injury occurs as a result of a trauma, as a result of a surgical procedure, as a result of patient positioning during surgery, as a result of a compression injury or a crush injury, as a result of a disease or condition relating to a loss of motor or sensory nerve function, as a result of a congenital anomaly, as a result of an amputation, as a result of complete or partial removal of an organ, tumor or tissue, as a result of a metabolic/endocrine complication, inflammatory disease, autoimmune disease, vitamin deficiency, infectious disease, toxin, exposure to organic metal or heavy metal, or administration of a medication or drug, or occurs as a result of a combination of any of these.

In various embodiments, a nerve injury comprises the loss of a segment of nerve. In various embodiments, a nerve injury comprises a nerve lesion of less than about 1 cm in length. In various embodiments, a nerve injury comprises a nerve lesion of at least 1 cm in length. In various embodiments, a nerve injury comprises a nerve lesion of at least about 3 cm in length. In various embodiments, a nerve injury comprises a nerve lesion of at least about 5 cm in length. In various embodiments, a nerve injury comprises multiple nerve lesions.

In various embodiments, one or more neurons are transplanted into a peripheral nerve, a cranial nerve or a spinal cord nerve of the subject. In various embodiments, one or more neurons are injected into a proximal nerve stump of the subject. In various embodiments, one or more neurons are transplanted in a delivery device that is secured to the proximal nerve stump. In various embodiments the delivery device may be any device secured to the proximal nerve stump and containing neurons. In various embodiments, the delivery devices is a nerve conduit, nerve wrap, or a nerve cap (i.e. sealed on one end with the open end in contact with the proximal stump). In various embodiments, the neurons in the delivery device preserve the regenerative capacity and prevent neuroma formation. After some period, the delivery device is surgically removed to allow for delayed nerve repair using a graft (autograph, acellular nerve allograft, conduit, TENG).

In various embodiments, one or more neurons are encapsulated in an extracellular matrix sheath and transferred into the delivery device. In various embodiments, one or more neurons are stretch-grown in culture, encapsulated in an extracellular matrix sheath, and transferred into the delivery device. In various embodiments, one or more neurons are pre-encapsulated in extracellular matrix, grown in culture, and then transferred into the delivery device. In various embodiments, one or more neurons are encapsulated in extracellular matrix and stored for at least about one month, at least about one week, at least about three days, at least about one day prior to transfer into a delivery device. In various embodiments one or more neurons are encapsulated in extracellular matrix, stored until needed and transferred to the delivery device immediately prior to implantation.

In various embodiments, one or more neurons are comprise of glutamatergic, GABAergic, sensory neurons, motor neurons or combinations thereof. In various embodiments, the one or more neurons are accompanied by Schwann cells, macrophages, fibroblasts, mesenchymal stem cells and/or myocytes. In various embodiments, the Schwann cells, macrophages, fibroblasts, mesenchymal stem cells or myocytes are implanted along at least a portion of the extracellular sheath encapsulating the one or more neurons.

In various embodiments, one or more neurons is derived from neuronal progenitor cells. In various embodiments, neurons may be derived from any neuronal progenitor cells known in the art. In various embodiments, neuronal progenitor cells are Spinal motor, cranial motor, peripheral sensory, and/or cranial sensory.

In various embodiments, one or more neurons is derived from stem cells. In various embodiments, stem cells are embryonic stem cells, in various embodiments stem cells are induced pluripotent stem cells. In various embodiments, the stem cells are spinal motor, cranial motor, peripheral sensory, and/or cranial sensory stem cells.

In various embodiments, one or more neurons, are engineered (e.g., transduced) to overexpress one or more neurotrophic factors, including, but not limited to GDNF, BDNF, VEGF, or NGF. In various embodiments, one or more neurons are supplemented with cells transduced to overexpress neurotrophic factors, including, but not limited to GDNF, BDNF, VEGF, or NGF. In various embodiments, the supplemental cells are Schwann cells, myocytes, fibroblasts, macrophages, and/or mesenchymal stem cells.

In various embodiments, the pro-regenerative capacity of a proximal nerve segment is maintained for at least about one week, at least about two weeks, at least about three weeks, at least about four weeks, at least about eight weeks, at least about twelve weeks, at least about 16 weeks, at least about six months at least about one year or indefinitely. In various embodiments, the pro-regenerative capacity of a proximal nerve segment is maintained until at least such time as proximal nerve axons regenerate across the nerve injury. In various embodiments the pro-regenerative capacity of a proximal nerve segment is maintained until at least such time as proximal nerve axons reinnervate distal targets.

In various embodiments, provided methods may further comprise a primary procedure for nerve repair that results in a greater degree of functional recovery following repair of PNI, as compared to the degree of functional recovery that occurs following the primary procedure alone. In various embodiments, provided methods may further comprise a primary procedure to preserve the regenerative capacity of a proximal nerve segment followed by a delayed nerve repair at a later time point that results in a greater degree of functional recovery following repair of peripheral nerve injury, as compared to the degree of functional recovery that occurs following a delayed repair alone. In various embodiments the improved degree of functional recovery is measured by recovery of sensation, motor function, and diminished neuropathic pain. In various embodiments, a provided method is conducted in the absence of any other nerve repair.

In various embodiments, provided methods may further comprise providing a neurotrophic factor, culture supernatant, or cells to the distal nerve segment. In various embodiments, the method does not comprise transecting a nearby healthy nerve or the repaired nerve.

In various embodiments, provided methods do not comprise transecting a nearby healthy nerve or the repaired nerve. In various embodiments, a distal site is less than least about 3 cm away from the site of injury.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

Materials and Methods

All supplies were from Invitrogen (Carlsbad, Calif.), BD Biosciences (San Jose, Calif.), or Sigma-Aldrich (St. Louis, Mo.) unless otherwise noted.

### Dorsal Root Ganglion Neuron Isolation

Dorsal root ganglia (DRG) were isolated from embryonic day 16 Sprague-Dawley rats (Charles River, Wilmington, Mass.). Briefly, timed-pregnant rats were euthanized, and pups were extracted through Caesarian section. Each fetus was removed from the amniotic sac and put in cold Leibovitz-15. Embryonic DRG explants were isolated from spinal cords and either plated directly into media or dissociated. For dissociation, explants were suspended in pre-warmed trypsin (0.25%)+EDTA (1 mm) at 37° C. for 45 min. Followed by the addition of neurobasal medium+5% FBS, the tissue was vortexed for at least 30 seconds and then centrifuged at 1000 rpm for 3 minutes. The supernatant was aspirated, and the cells were resuspended at 5×10⁶ cells/mL in media. Media for both dissociated cells and explants consisted of neurobasal medium+2% B-27+400-500 μm L-glutamine+1% fetal bovine serum (Atlanta Biologicals)+2.0-2.5 mg/mL glucose+10-20 ng/mL 2.5S nerve growth factor, and a mitotic inhibitor formulation of 10 mM 5-fluoro-2′-deoxyuridine (5FdU) and 10 mm uridine to encourage non-neuronal cell elimination.

### Nerve Repair Preparation

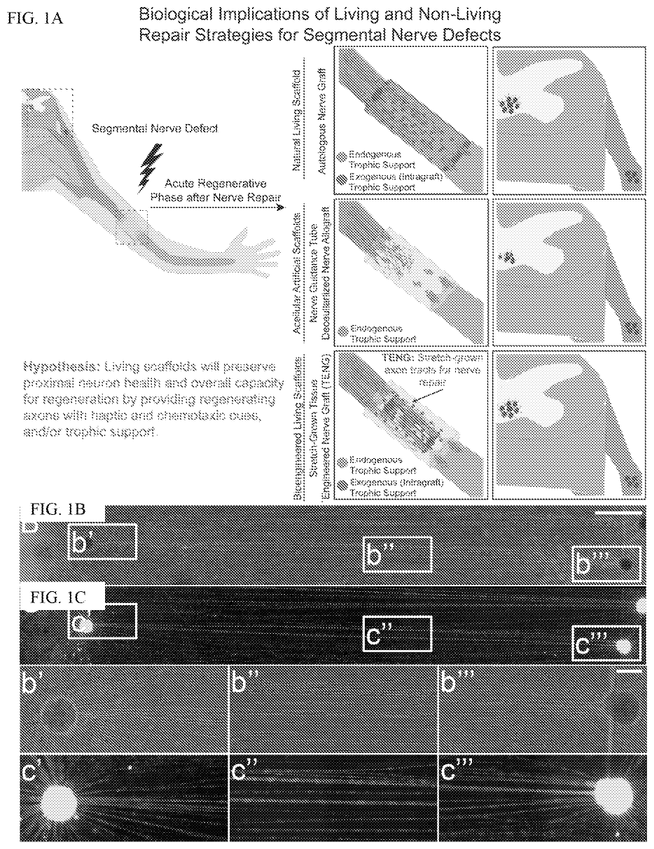

TENGs were fabricated by stretch-growing DRG explants plated in custom-fabricated mechanical elongation bioreactors as previously described. Briefly, DRGs were plated in two populations along the interface of an aclar “towing” membrane treated with poly-D-lysine (20 ug/mL) and laminin (20 μg/ml), resulting in a separation of approximately 500 Cells were transduced with an AAV viral vector (AAV2/1.hSynapsin.EGFP.WPRE.bGH, UPenn Vector Core) to produce GFP expression in the neurons. At 5 days in vitro (DIV) the cells were incubated overnight in media containing the vector (3.2×10¹⁰ genome copies/ml) and the cultures were rinsed with media the following day. Over 5 DIV, axonal connections were formed between the two populations. The populations were then gradually separated over the course of 6 days using a stepper motor system to displace the cells at a rate of 1 mm per day for 2 days and then 2 mm per day for 4 days, until the axons spanning them reached a total length of 1.0 cm as previously described (FIGS. 1B-1C). Fresh, pre-warmed media was used to replace the culture media every 2-3 days. After 11-13 DIV, once the stretched constructs had reached the desired length, their health was assessed. Constructs appropriate for transplant were removed from the incubation chambers and embedded in a collagen-based matrix (80% v/v) in Minimum Essential Media (MEM 10X) and sterile cell culture grade water supplemented with NGF (2.5S, 0.05 ng/mL). After gelation at 37° C., embedded cultures were gently removed and placed within a 1.2 cm long absorbable collagen nerve guidance tube (Stryker NeuroFlex™) and transplanted into a rat sciatic nerve injury model. For the NGT+DRG group, two populations of whole DRG explants (10 DRG in each row) were plated 1 cm apart on an ACLAR membrane to resemble the environment within the mechanobioreactor. Axonal connections were allowed to form for 5 DIV as is done prior to initiation of mechanical tension for stretch grown constructs, and the cells were encapsulated in the collagen matrix described above and transferred into a 1.2 cm NGT for transplantation. The NGT control group received the same collagen matrix within the conduit.

### Surgical Procedure

Nerve regeneration was evaluated in vivo in a 1 cm rodent sciatic nerve injury model at 2 weeks following injury. A total of 20 male Sprague-Dawley rats were assigned to 5 groups: naïve (n=5), autograft (n=3), nerve guidance tube (NGT) (n=4), NGT containing disorganized DRG neurons (NGT+DRG; n=4), and TENGs (n=4). In addition, a total of 10 naïve rats were used to validate the fluorescent retrograde tracing methodology compared to conventional immunohistochemistry. Rats were anesthetized with isoflurane and the left hind was cleaned with betadine. Meloxicam (2.0 mg/kg) was given subcutaneously and bupivacaine (2.0 mg/kg) was administered along the incision line subcutaneously. The gluteal muscle was separated to expose the sciatic nerve exiting the sciatic notch. The sciatic nerve was carefully dissected to its trifurcation. The sciatic nerve was sharply transected 5 mm distal to the musculocutaneous nerve and a 1 cm nerve injury was made. For autograft repairs, a reverse-autograft technique was used. For NGT, NGT+DRG, and TENG repairs, the nerve stumps were carefully inserted into each end of the nerve guidance tube with an overlap of 1 mm, and the epineurium was secured to the tube using 8-0 non-absorbable prolene sutures. The NGT+DRG repair contained DRG neurons embedded in collagen and NGT repairs contained collagen only as described above. The surgical site was closed with 3-0 absorbable vicryl sutures and skin staples. Animals were recovered and returned to the vivarium.

### Dye Application

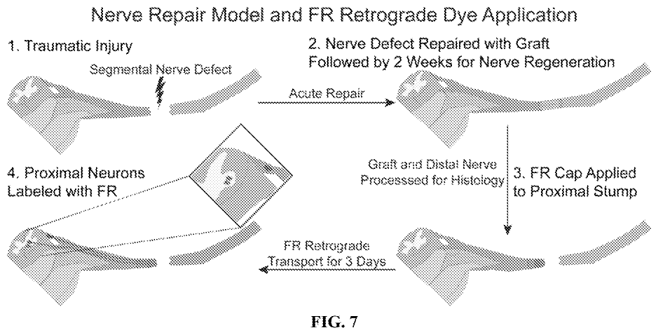

At the terminal time point retrograde dye was applied to the nerve. In brief, a nerve cuff was fashioned by capping silicon tubing (A-M Systems, 807600, 0.058″×0.077″×0.0095″) with PDMS (Fisher Scientific, Sylgard) that was trimmed to a length of 4 mm. The resulting nerve cuffs were stored in 70% ethanol until used. Prior to transplantation, the cuffs were rinsed in PBS and dried using a Kimwipe. In this study, retrograde dye transportation was evaluated following acute nerve regeneration at 14 days post repair (FIG. 7). In a subset of animals, retrograde dye transportation was assessed following chronic nerve regeneration at 16 weeks post repair.

At 14 days after the initial repair procedure, the surgical site was re-exposed and the sciatic nerve was harvested, beginning 5 mm proximal to the repair zone, and immersion-fixed in formalin. A 2 mm by 2 mm piece of Kim wipe was soaked in 30% Fluoro-Ruby (FR; EMD Millipore, AG335) and placed inside the silicon nerve cuff toward the bottom. The silicon cuff was compressed, thereby creating a negative pressure vacuum, and the cuff was placed at the face of the proximal nerve stump. By slowly releasing the compression on the cuff, the negative pressure allowed for the sciatic nerve to seal within the cuff. Tisseel Fibrin Sealant (Baxter, 1504514) was applied around the cuff to fix the proximal nerve and cuff in place. The surgical site was closed using 3-0 absorbable vicryl sutures and skin staples, and the animal returned to the vivarium for 3 days of FR exposure (FIG. 7).

### Nerve Regeneration and Schwann Cell Infiltration Across the Repair Zone

Formalin-fixed, frozen nerve sections were rinsed in PBS (3×5 min) and blocked for 1 hour in blocking solution (PBS with 4% normal horse serum and 0.3% Triton X-100). Primary antibodies diluted in blocking solution were then applied and incubated overnight at 4° C. Mouse anti-phosphorylated neurofilament (SMI-31, 1:1000, BioLegend Cat #801601) and mouse anti-nonphosphorylated neurofilament (SMI-32, 1:1000, BioLegend Cat #701601) were used to identify axons; rabbit anti-S100 Protein Ab-2 (S100, 1:500, Thermo Scientific Cat #RB-044-A) was used to identify Schwann cells. Following incubation, slides were rinsed in PBS (3×5 min) and secondary antibodies prepared in blocking solution were applied for 2 hours at room temperature: donkey anti-mouse 568 (Alexa Fluor® 568, 1:500, Thermo Scientific Cat #A10037) and donkey anti-rabbit 647 (Alexa Fluor® 647, 1:500, Thermo Scientific Cat #A31573). Sections were then rinsed in PBS (3×5 min), mounted with Fluoromount-G® (Southern Biotech Cat #0100-01) and coverslipped. Images were obtained with a Nikon MR confocal microscope (1024×1024 pixels) with a 10× air objective and 60× oil objective using Nikon NIS-Elements AR 3.1.0 (Nikon Instruments, Tokyo, Japan).

### Spinal Cord Tissue Acquisition

Animals were transcardially perfused with 10% formalin and heparinized 0.9% NaCl. L4/L5 DRGs were extracted. Spinal cord T12-L6 region was extracted en bloc. All samples were fixed in paraformaldehyde overnight then placed in 30% sucrose for 48 hours. Full en bloc spinal cord was embedded in OCT then frozen. Tissue orientation was preserved with the use of tissue dye. Spinal cord samples were sectioned at 500 μm on a microtome cryostat and examined briefly under Nikon A1RSI Laser Scanning confocal microscope paired with NIS Elements AR 4.50.00 to screen sections with visible FR in the ventral horn. Spinal cord sections and DRGs with positive FR signal were stored in PBS for 24 hours.

### Spinal Cord Optical Clearing

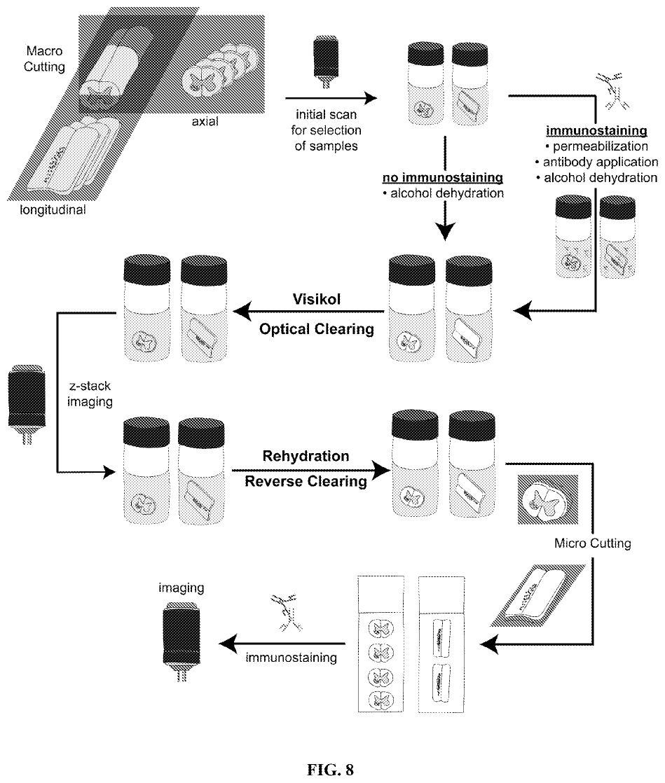

Spinal cords were sectioned into 500 μm thick blocks and DRGs were optically cleared using the Visikol method and all washes were conducted at 15-minute time intervals unless otherwise stated (FIG. 8). Spinal cord sections were washed in increasing concentrations of methanol (50%, 70%, and 100%) and stored for 12 hours in 100% methanol. Samples were then exposed to 20% DMSO/methanol followed by decreasing concentrations of methanol (80% and 50%). Next, samples were washed with PBS followed by PBS/0.2% Triton X-100. Next, samples were incubated in penetration buffer (0.2% Triton X-100, 20% DMSO, and 0.3M glycine in 1×PBS), then blocking buffer (0.2% Triton X-100, 6% NHS, and 10% DMSO in 1×PBS) at 37° C. for 19 hours each. Samples were rinsed twice in washing buffer, then exposed to primary antibodies (1:500 Rabbit NeuN) in antibody buffer (0.2% Tween, 10 μg/mL Heparin, 3% NHS, and 5% DMSO in 1×PBS). After incubation in primary antibodies, samples were rinsed ten times in washing buffer (0.2% Tween and 10 μg/mL Heparin in 1×PBS), followed by exposure to secondary antibodies (1:500 Donkey anti-rabbit 647). Samples were again rinsed ten times in washing buffer. Finally, samples were exposed to increasing concentrations of methanol (50%, 70%, and 100%) at three rinses each while incubated at 37° C. The samples were then incubated in Visikol 1 for 24 hours, then Visikol 2 for 48 hours. For all DRG samples, these samples were placed in Visikol 1 after the initial ascending methanol steps.

### Quantification of Ventral Horn and Dorsal Root Ganglia

For conventional immunohistochemistry: naïve spinal cords were harvested 3 days following FR application in order to validate the optical clearing technique. Briefly, samples were stored in 30% sucrose for 24 hours or until saturation for cryoprotection, embedded in optimal cutting temperature compound (OCT), and frozen in −80° C. isopentane. Axial sections were taken using a cryostat microtome (30 μm thick) and stained for NeuN (1:500 Rabbit NeuN, 1:500 donkey anti-rabbit 647). Slides were imaged at 20× using a Nikon A1RSI Laser Scanning confocal microscope paired with NIS Elements AR 4.50.00, taking z-stacks at 5 μm intervals. FR and NeuN cell counts were quantified from maximum projections. The Abercrombie correction for cell quantification was applied based on section thickness and an estimated cell size of 30 μm.

For optical cleared tissue: samples were placed in glass bottom well plates and immersed fully in Visikol 2. Tissue was then imaged using Nikon A1RSI Laser Scanning confocal microscope under 10× air objective. All images were taken using a z-stack at 5 μm steps with laser settings optimized for naïve tissue. Images were analyzed using ImageJ FIJI software. Maximum projections of 100 μm were generated and all cells within the ventral horn (L4-L6) of the spinal cord and DRG (L4, L5) were quantified by drawing line ROI through background and the entirety of the cell body. Using a custom MATLAB script, the intensity value for each cell body was calculated with the following formula:

$$\frac{maximum intensity - background intensity}{background intensity}$$

### Functional Assessment

At 16 weeks post repair, compound muscle action potential (CMAP) was assessed to evaluate functional regeneration. Animals were re-anesthetized and the graft was re-exposed, A bipolar subdermal electrode was placed in the tibialis anterior and the nerve was stimulated (biphasic; amplitude: 0-10 mA; duration: 0.2 ms; frequency: 1 Hz) using a handheld bipolar hook electrode (Rochester Electro-Medical, Lutz, Fla.; #400900) 5 mm proximal to the repair zone. The supramaximal CMAP recording was obtained and averaged over a train of 5 pulses (100× gain and recorded with 10-10,000 Hz band pass and 60 Hz notch filters).

### Statistical Analysis

For conventional and optical cleared quantification: the mean and standard error were calculated for cell counts and compared using a parametric one-way ANOVA with multiple comparisons. To compare the number of FR⁺ cells across each experimental group, a parametric ANOVA test with multiple comparisons was conducted with an alpha value of 0.05. The intensity of every cell per animal was log transformed to fit the naïve group data to a normal distribution. Frequency distributions for each experimental group were calculated with a bin length of 0.1 for MN and DRG. These frequency distributions were normalized by dividing each bin frequency by the experimental group sample size. The mean intensity of each animal was averaged, and experimental groups were compared using a nonparametric Kruskal-Wallis ANOVA with multiple comparisons. A linear regression was generated between number of FR⁺ cells and the transformed mean intensity value.

### Results

### Repair Zone Analysis

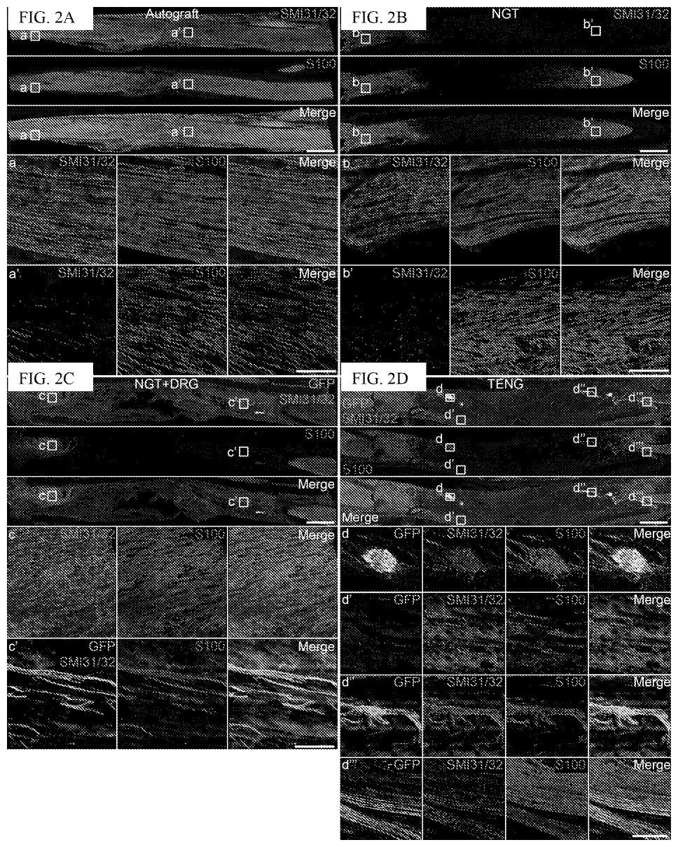

A 1-cm segmental defect was created in the sciatic nerve of rats, with immediate surgical repair using a reverse-autograft, an NGT containing a collagen-based ECM, an NGT containing disorganized DRG neurons throughout the ECM (“NGT+DRG”), or custom-fabricated TENGs featuring longitudinally-aligned axonal tracts projecting from DRG neurons within ECM. To evaluate acute regeneration processes across the repair zones, at 2-weeks post-repair longitudinal sections from rat sciatic nerve grafts were immunostained with SMI31/32 and S-100 to visualize axons and Schwann cells, respectively. Axonal infiltration from the proximal region into the graft region was observed across all groups (FIGS. 2A-2D). Regenerating axons and Schwann cell infiltration were observed at higher magnification in longitudinal sections of the graft region. Implanted DRG expressing GFP survived transplantation in the NGT+DRG group (FIG. 2C). Moreover, TENG neurons also survived transplantation and extended axons into the distal nerve, and integrated with host Schwann cells and axons, enabling axon-facilitated axonal regeneration (AFAR) across the graft zone (FIG. 2D).

Neuronal Labeling Validation in Optically Cleared Sections from Naïve Tissue

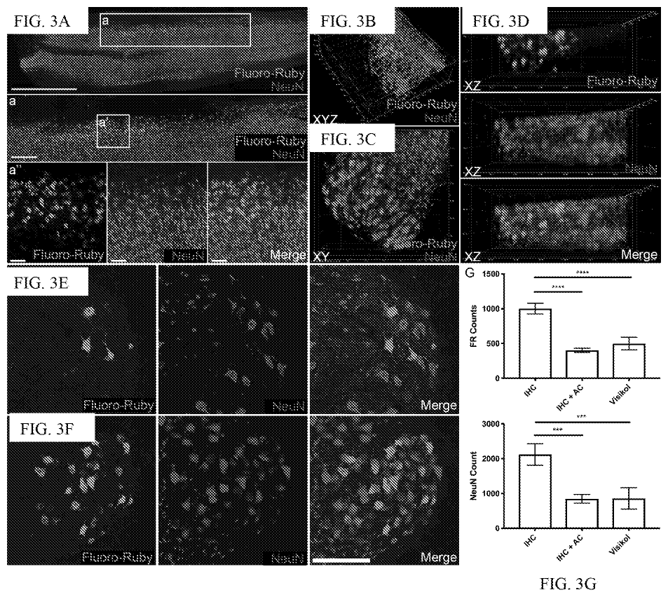

To validate the optical clearing technique and quantification methodology used in subsequent analyses, FR and NeuN were visualized in 500 μm thick naïve spinal cord blocks. FR labeled cells were clearly visualized within optically cleared spinal cord stained for NeuN in both axial and longitudinal blocks and adequate antibody penetration was confirmed in the volumetric reconstruction of the Z-stack images across various X-Y-Z planes (FIGS. 3A-3G). FR and NeuN cells were quantified in maximum projection z-stack images for 500 μm thick sections of spinal cords and 30 μm thick frozen sections. Conventional IHC cell counts were significantly higher for FR and NeuN counts than the IHC quantification with the Abercrombie correction (IHC+AC) or Visikol HISTO sectioning. No statistical significance was found for FR or NeuN quantification between the IHC+AC and Visikol HISTO methods (p>0.05).

### Ventral Horn Retrograde Labeling Analysis Following PNI Repair

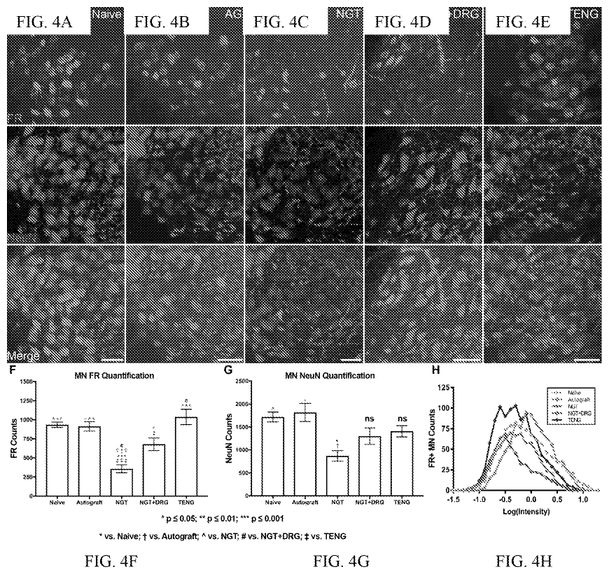

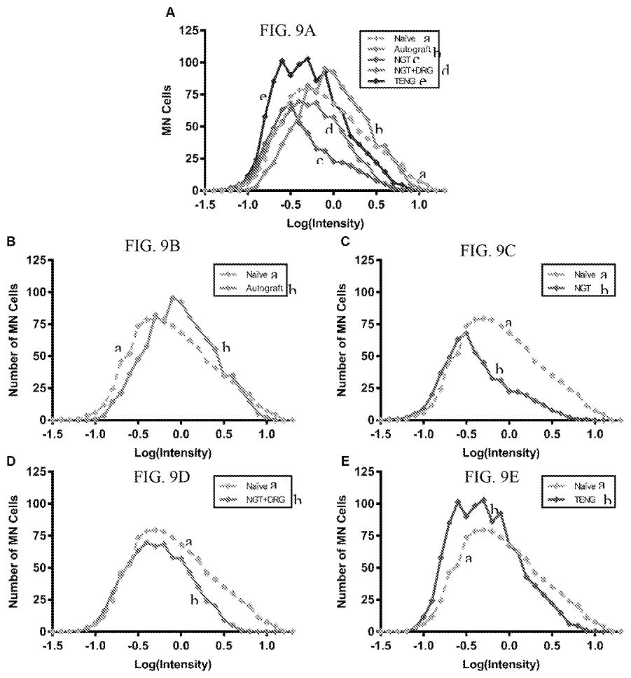

The retrograde dye FR was applied to the nerve segment proximal to the repair zone at 14 days post-repair, and the presence of FR in proximal host neurons was assessed 3 days thereafter as a surrogate for neuronal health and active regeneration. In the spinal cord, TENG repairs exhibited a similar number of FR⁺ labelled MN cell bodies (1038.0±100.5) as naïve animals (935.0±35.4) and autograft repairs (914.7±35.4) (p>0.05) (FIG. 4A-4H). TENG repairs also exhibited a significant increase in FR⁺ MNs compared to NGT (357.3±52.3, p<0.001) and NGT+DRG repairs (678.8±82.6, p<0.05). NGT repairs exhibited a significant decrease in the number of FR⁺ MN cell bodies as compared to naïve, autograft and TENG repairs (p<0.001 each), as well as NGT+DRG repairs (p<0.05) (FIG. 4F). The density of NeuN⁺ was also quantified within the ventral horn of the spinal cord. NGT NeuN counts were significantly less than naïve and autograft NeuN counts (p<0.05), while NGT+DRG and TENG NeuN counts were not statistically different from naïve animals (p>0.05) (FIG. 4G). The intensity of FR uptake per neuron was also quantified as a further metric of neuronal health. NGT repairs exhibited a significantly diminished number of high FR intensity cells than the naïve group. Autograft repairs showed a rightward shift in FR intensity as compared with naïve group while TENG repairs showed a similar FR intensity profile as naïve repairs (FIG. 4H; See FIGS. 9A-9F for a breakout of various distributions per experimental group).

### Dorsal Root Ganglia Retrograde Labeling Analysis Following PNI Repair

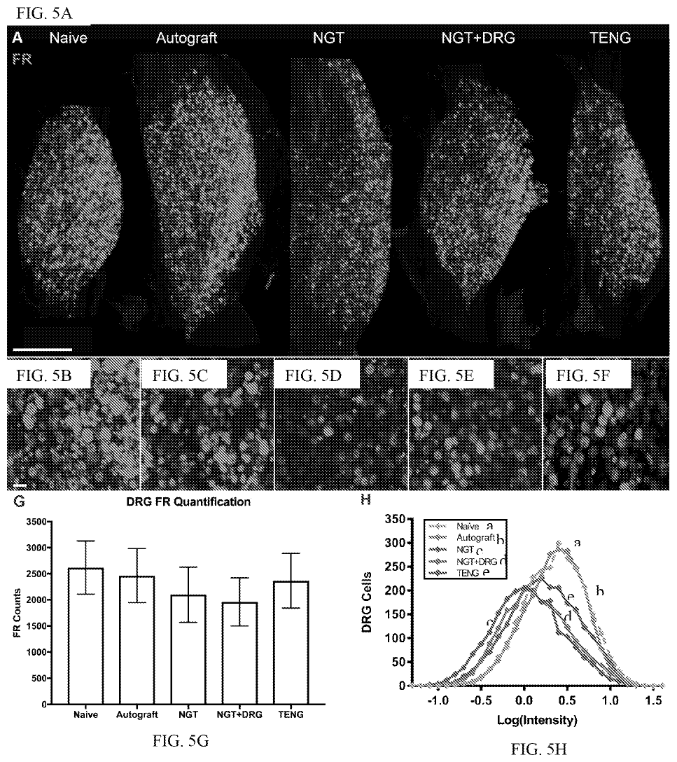

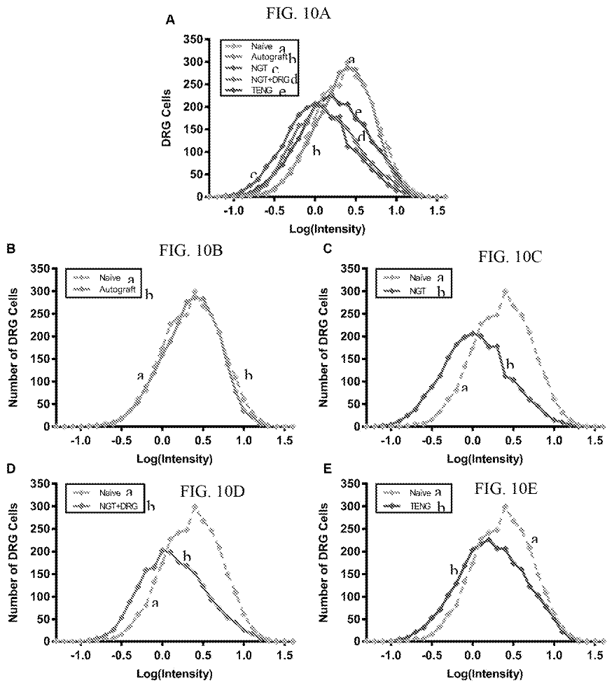

Similar to the MN analysis of retrograde FR labeling, the density and intensity of FR labeling in L4 and L5 DRG were also assessed. There were no statistical differences in the number of FR⁺ cells across experimental groups in the L4 and L5 DRG regions (FIGS. 5A-5H). The intensity of FR uptake per neuron was again quantified as a metric of neuronal health. Autograft repairs exhibited a similar FR intensity profile as naïve animals, while NGT, NGT+DRG and TENG repairs showed a downward shift in FR intensity profile as compared with the naïve group (FIG. 5H; See FIGS. 10A-10E for a breakout of various distributions per experimental group).

### FR Uptake and Functional Assessment at 16 Weeks Following PNI Repair.

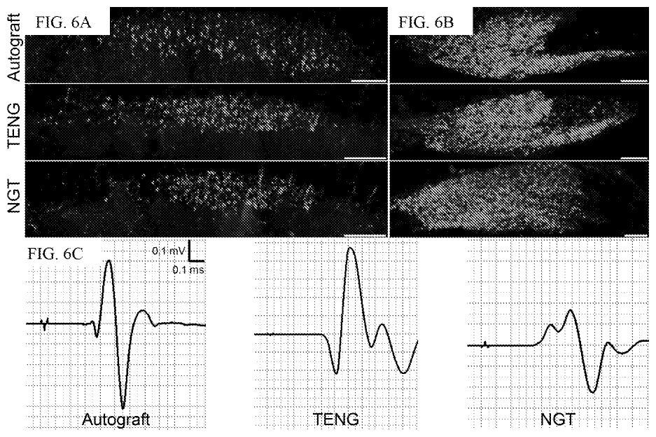

Structural and functional measures were assessed at 16 weeks following PNI repair to relate acute changes in retrograde neuronal labeling with more chronic outcomes. A qualitative assessment of a subset of animals at 16 weeks post repair revealed similar numbers of FR labeled spinal motor neurons following TENG and autograft repair, as compared to a reduced number of labeled neurons following NGT repair (FIGS. 6A-6C). No differences were observed in the labeling of DRG neurons between groups. CMAP recordings were obtained in all animals at 16 weeks post repair, revealing that the amplitude of the response was consistently much greater following TENG or autograft repair than following NGT repair. The inclusion of these functional recovery measures in the current study provides further context and a neurobiological mechanism that likely at least partially explains decreased CMAP following an acellular repair strategy as compared to enhanced CMAP following living scaffold repair strategies (i.e. autografts and TENG repairs).

PNI recovery is a race against time since regenerating axons have a limited period to reach distal end targets before the pro-regenerative distal nerve environment loses its capacity to support regeneration. Here, various approaches have been developed to facilitate nerve regeneration across a graft region and ultimately sustain the pro-regenerative environment in the distal segment. However, there has been limited focus on the ability for a graft to influence the intrinsic regenerative capacity of proximal neurons. To address this gap, the current study compliments our previous efforts for TENG development and implementation by highlighting an additional mechanism by which this class of tissue engineered “living scaffolds” may lead to an improved time course and extent of functional recovery. Of note, this mechanism of preserving proximal neuronal health and regenerative capacity was superior using TENGs as compared to acellular repair strategies, such as commonly used NGTs, and resulted in a commensurate level of increased functional recovery. To the best of our knowledge, this is the first study to investigate the effect of different peripheral nerve repair strategies on proximal neuron health and regenerative capacity.

In this study, FR expression in host spinal cord motor neurons and DRG neurons was quantified as a surrogate marker for retrograde transport capability and overall neuron health. At 2 weeks post repair, a similar number of FR⁺ cells was found in the ventral horn in animals repaired using TENGs or autografts, and both matched that found in naïve animals. In contrast, NGT repairs resulted in >65% reduction in FR⁺ neurons as compared to TENG repairs, likely due to acellular NGTs not providing adequate neurotrophic and anisotropic structural support. Furthermore, the average fluorescent intensity of the FR⁺ spinal MNs in the NGT repair group was significantly less than that found in naïve animals. In addition, nerves repaired with NGTs seeded with disorganized DRG populations exhibited a modest increase in the level of retrograde transport compared to the NGT group, potentially indicating the benefit of a cellular-derived trophic component alone for PNI repair. The benefit of a living cellular component in a nerve graft was further corroborated with evidence of Schwann cell infiltration surrounding transplanted cells within the graft zone. Although the reduction in FR⁺ labeling in the ventral spinal cord of the NGT group compared to NGT+DRG groups indicates the advantage of living cells within the graft, the extent of FR⁺ cells in the NGT+DRG group was significantly less that that found following TENG or autograft repairs. This suggests the dual importance for nerve grafts to supply trophic factors while simultaneously presenting a biomimetic, anisotropic structural organization.

This study also assessed the presence of NeuN⁺ neurons in the spinal cord and DRG following various PNI repair strategies. Historically, NeuN has been considered a reliable mature neuron marker that is highly conserved across species and useful for quantifying neuronal density (e.g., to indirectly assess neuronal loss). However, recent work has shown that NeuN antibody specificity generally depends on phosphorylation state. Anti-NeuN binds to phosphorylated NeuN within neuronal nuclei, therefore, loss of NeuN immunoreactivity may indicate a stress response but not necessarily neuronal death. In this study, NeuN counts in the spinal cord were similar between naïve animals and autograft repairs, and neither were significantly different from TENG or NGT+DRG repairs. In contrast, fewer NeuN-labeled spinal MNs were visualized following NGT repair. The implication of these findings is unclear as NeuN expression has not been well characterized with respect to injury severity or repair strategy. However, facial nerve axotomy has been reported to result in rapid and protracted decrease in NeuN expression compared to transient NeuN loss after facial nerve crush injury. In addition, chronic nerve axotomy induced by avulsion injury was shown to result in a rapid and persistent loss of NeuN expression in the spinal cord ventral horn; however, motor neuron death progressed over the course of 4 weeks. The rapid loss of NeuN expression at this acute time point is likely associated with biochemical disturbances and molecular changes to the injured cell body resulting from the axonal disconnection to the distal structures and deprivation of Schwann cell- and/or muscle-derived neurotrophic support. Therefore, injury severity and grafting strategy may have a major role in NeuN labeling and/or expression.

Whether the loss of NeuN expression is persistent or transient may be dependent on the injury severity. After crush nerve injury, the spared underlying nerve tissue provides a robust cache of neurotrophic support from the distal structures, enabling more rapid regeneration to the end target. In contrast, severe nerve injury, such as chronic nerve axotomy or root avulsion, deprives the cell body of crucial neurotropic factors secreted by the distal structures. These mechanisms may also play a significant role in differences in NeuN expression after segmental nerve repair. Following autograft repair, trophic factors secreted by Schwann cells within the donor nerve may help to sustain the injured proximal neurons. In contrast, NGTs lack endogenous Schwann cells, which may deprive the proximal neurons of neurotrophic factors and result in decreased NeuN expression. Following NGT repair, acute loss of NeuN expression could be transient and reverse as the regenerating axons interact with pro-regenerative Schwann cells moving in from the distal nerve stump; however, our findings at 16-weeks post repair suggest that this NeuN loss may be permanent, and thus represent neuronal cell death. In contrast, similar to autografts, the DRG+NGT and TENG groups likely preserved NeuN expression by providing regenerating axons with trophic factor support; however, the exact mechanism remains unclear as these constructs were not supplemented with Schwann cells. Overall, our findings warrant follow-on studies to determine whether decreased NeuN expression following segmental nerve defects provides an indication of neuronal cell health or if loss of expression is generally associated with permanent neuronal cell death. If the decreased NeuN expression is transient, then these findings potentially suggest strategies lacking trophic support may result in a large number of unhealthy proximal neurons during a crucial period for regeneration. Moreover, the ability for living scaffolds to sustain neuronal health at an acute time point further suggests the importance for maintaining overall regenerative capacity to enhance the rate and extent of functional recovery.

Although there were stark differences in the level of FR and NeuN expression in the ventral spinal cord, there did not appear to be any significant change in DRG neurons based on FR or NeuN expression across any of the experimental groups. These results are consistent with previous studies that found no change in the number of retrogradely labeled DRG neurons at two weeks following sural nerve axotomy. Previous studies also corroborate this finding, demonstrating that sensory neurons are more resilient and regenerate more robustly than motor neurons at early time points following nerve axotomy. More extensive studies are necessary to elucidate the intrinsic gene expression patterns and physiological pathways in DRG that make these neurons less dependent in extrinsic factors for injury and regeneration responses.

In the current study, retrograde transport was used as a surrogate marker for neuronal cell health and potential regenerative capacity, with quantitative measurements made of the density of FR⁺ cells as well as the per-cell intensity of FR expression. Indeed, retrograde signaling is necessary to increase protein synthesis and enable growth cone extension. While there is substantial evidence that retrograde transport is modulated through varying levels of neuronal cell health, other mechanisms, such as the rate of axonal resealing, might modulate the amount of FR transported to the neuronal cell body. Previous studies have shown evidence that axon diameter may also influence rate of microtubule-based retrograde transport. In order to conclusively implicate active retrograde transport as the modulator of observed changes in FR expression, it may be of interest in future experimental designs to include a control repair group that is exposed to a retrograde transport inhibitor, such as Ciliobrevin D. In addition, the per-cell intensity of FR expression was used as a representative marker of the extent of retrograde transport and therefore as an indirect marker of neuronal health. Of note, the fluorescent intensity of the cell body was calculated using the subtraction of the background intensity from the maximum intensity relative to the background intensity based on previous methodology. However, in future analyses, alternative methodology such as comparing the mean pixel intensity across cell bodies and/or factoring in the somata volume may offer additional useful information on neuronal health.

Living scaffolds may provide a sustained bolus of numerous pro-regenerative neurotrophic factors that support regenerating axons (and hence the neurons that project these axons) and ultimately facilitate functional recovery. In this study, we found TENGs and autografts had a similar degree of healthy neurons at an acute time point post-injury and similar levels of functional recovery chronically. Both autograft and TENG repairs resulted in substantially improved metrics of acute regeneration and chronic recovery than NGT repairs. It is important to note that these results should be interpreted in context, as some metrics were not statistically significant yet there were large differences in means, suggesting that although these findings are promising, further optimization may improve the consistency of improvements in neuronal health and ultimately functional recovery. In addition, future studies are necessary to more directly investigate whether early recovery of neuronal health also improves the capacity for muscle reinnervation. To further understand regenerative capacity for clinical applicability, future studies might also include additional cell markers to provide additional insight into the neurons that are actively regenerating toward the end targets. The varying expression profile of certain transcriptional factors, such as ATF-3 and C-JUN, following nerve injury, during regeneration, and until reinnervation could be combined with FR expression data to provide greater insight into the duality between regeneration and neuronal cell health.

Although TENGs have demonstrated the potential for preserving neuronal cell health, further optimization is necessary to tailor the repair strategy for specific injuries. For example, sensory nerve autografts are typically used to repair all injuries, including primarily motor as well as mixed motor-sensory nerves. However, there has been some evidence that suggests motor nerve autografts may further increase functional recovery. Therefore, it might be useful to develop modality-specific TENGs comprise sensory, motor, or mixed motor-sensory neurons/axons to further enhance the regenerative capacity across multiple types of nerves. However, in this study, TENGs comprised of DRG neurons/axon tracts were shown to maintain neuronal cell health in MN regions of the spinal cord.

To date, most commercially available NGTs are empty conduits which lack neurotrophic and anisotropic support (e.g., NeuroTube®, poly-glycolic acid, Baxter/Synovis; NeuraGen®, collagen, Integra Life Sciences; Neuroflex™, collagen, Stryker), although there has recently been the introduction of an NGT filled with collagen containing aligned channels to provide anisotropic guidance (i.e., Nerbridge®, Toyobo). Numerous previous studies have reported that repairs using an NGT resulted in diminished functional recovery compared to an autograft repair, even in gap repairs less than 1.5 cm. Our study corroborated this by demonstrating that an acellular graft with solely structural isotropic support from collagen was unable to support proximal cell survival and regenerative capacity as compared to other grafts. NGTs are therefore inadequate clinical vessels for supporting effective, healthy, and functional regeneration in all cases. Acellularized nerve allografts (ANAs) were developed as an alternative non-living scaffold repair strategy that provided regenerating axons with the nerve architecture and haptic cues similar to an autograft repair. However, ANAs lack a cellular component and the necessary trophic support for sustained nerve regeneration, thus likely minimizing regenerative capacity and the ultimate level of functional recovery.

Recent efforts have aimed to develop biomimetic materials and controlled release systems to offer alternative strategies to acellular NGTs by adding biologically-active structural proteins and soluble factors, such as ECM, NGF, BDNF, and GDNF. These enhanced grafts may prove effective in some instances of neural regeneration, including promising results showing host regeneration following long gap repairs. However, despite these promising engineering feats, even with the addition of biologically-active filler, acellular grafts remain unable to address overall shortcomings in nerve regeneration, potentially due to limitations in the number of factors delivered, a non-optimal presentation of these factors, and/or lack of sufficient temporal and spatial control of their release. However, undesirable results have also been reported using over-engineered cellular grafts, providing a prime example of the “candy store effect”. This effect was observed following the supplementation of NGTs with engineered Schwann cells over-expressing GDNF. Rather than promoting nerve regeneration with a strong chemoattractant for regenerating axons, the neurite outgrowth migrated to the site of GDNF release and did not move past this site of application—effectively countering the intended process of regeneration. As our data demonstrated, even by supplementing NGTs with disorganized DRGs, these grafts were still unable to provide signals sufficient to improve motor neuron cell health and support axon regeneration across the graft. Interestingly, others have shown that neurons directly transplanted into the distal nerve have improved muscle recovery in short-gap, long-gap, and delayed nerve repairs, possibly by providing direct structural integration and improving neurotrophic support. However, just as advantageous structural attributes alone (e.g., alignment) are not enough for maximal regenerative capacity, a solely living component absent structural anisotropy also appears to be insufficient. For this reason, autografts remain the only readily accessible living scaffold strategy currently available in a surgeon's armamentarium for peripheral nerve reconstruction that provides the native architecture and cellular support necessary for accelerating axonal regeneration, sustaining neuronal survival, and maximizing functional recovery. However, autografts have inherent shortcomings, including donor site morbidity and limited availability of donor nerve for long gap nerve repair and/or polytrauma resulting in multiple nerve injuries.

As a potential complimentary alternative to autografts, TENGs have continued to demonstrate the potential to overcome challenging limitations in nerve repair. Our group has previously reported that TENGs utilize a previously unknown mechanism described as “axon-facilitated axonal regeneration” to enable accelerated axonal regeneration and functional recovery compared to commercially-available NGTs, at levels equivalent to autograft repairs. Notably, TENG axons also penetrate the host distal nerve segment and “babysit” the distal Schwann cells as host regenerating axons cross the segmental defect—a mechanism not possible with autograft repairs. In the current study, we have shown TENGs also preserve the regenerative capacity of the proximal neuron populations within the spinal cord, thereby potentially increasing the ceiling for host regeneration and functional recovery (FIG. 1A). In this study, TENG repairs resulted in a greater number of motor neurons maintained following PNI as compared to NGT repairs. In fact, within these motor neuron regions of the spinal cord, TENGs preserved FR and NeuN expression comparable to autograft repairs and naïve animals. Furthermore, we found a dramatic reduction in neuronal cell health following NGT repairs, which has been corroborated by clinical reports demonstrating poor functional recovery. Although a qualitative assessment of functional recovery was performed to understand the chronic implications of the acute measures, further investigation is necessary to establish the importance of early preservation of proximal neurons and regenerative capacity on the rate and extent of functional recovery.

### CONCLUSION

This study demonstrates that early surgical intervention with living scaffolds, such as autografts or TENGs, may preserve host spinal cord motor neuron health and regenerative capacity following acute segmental nerve repair. By preserving the regenerative capacity, living scaffolds may increase the potential ceiling for functional recovery compared to nonliving scaffolds. Therefore, TENGs may represent a promising technology for peripheral nerve repair by facilitating regeneration across segmental defects and providing crucial acute neurotrophic support necessary for successful functional recovery without the need for sacrificing an otherwise healthy donor nerve.

### Enumerated Embodiments

Embodiment 1. A method for maintaining the health of motor neurons in the spinal cord and the pro-regenerative capacity of a proximal nerve segment subsequent to a nerve injury in a subject in need thereof, the method comprising transplanting a stretch-grown tissue engineered nerve graft (TENG) into a proximal site contacting the proximal nerve segment.
Embodiment 2. The method of Embodiment 1, wherein the subject is a mammal.
Embodiment 3. The method of Embodiment 2, wherein the subject is a human.
Embodiment 4. The method of Embodiment 1, wherein the nerve injury comprises an injury to a peripheral or cranial nerve of a subject.
Embodiment 5. The method of Embodiment 1, wherein the nerve injury comprises an injury to the spinal cord of a subject.
Embodiment 6. The method of Embodiment 1, wherein the nerve injury comprises the loss of a segment of nerve.
Embodiment 7. The method of Embodiment 1, wherein the nerve injury comprises a nerve lesion of less than about 1 cm in length.
Embodiment 8. The method of Embodiment 1, wherein the nerve injury comprises a nerve lesion of at least about 1 cm in length.
Embodiment 9. The method of Embodiment 1, wherein the nerve injury comprises a nerve lesion of at least about 3 cm in length.
Embodiment 10. The method of Embodiment 1, wherein the nerve injury comprises a nerve lesion of at least 5 cm in length.
Embodiment 11. The method of Embodiment 1, wherein the nerve injury comprises multiple nerve lesions.
Embodiment 12. The method of Embodiment 1, wherein the pro-regenerative capacity of the proximal nerve segment is maintained for at least about 16 weeks.
Embodiment 13. The method of Embodiment 1, wherein the pro-regenerative capacity of the proximal nerve segment is maintained until at least such time as proximal nerve axons regenerate across the nerve injury.
Embodiment 14. The method of Embodiment 1, wherein pro-regenerative capacity of the proximal nerve segment is maintained until at least such time as proximal nerve axons reinnervate distal targets.
Embodiment 15. The method of Embodiment 1, wherein the method further comprises a primary procedure for nerve repair.
Embodiment 16. The method of Embodiment 15, wherein the method results in a greater degree of functional recovery following repair of PNI, as compared to the degree of functional recovery that occurs following the primary procedure alone.
Embodiment 17. The method of Embodiment 1, wherein the method is conducted in the absence of any other nerve repair.
Embodiment 18. The method of Embodiment 1, further comprising providing a neurotrophic factor, culture supernatant, or cells to the proximal nerve segment.
Embodiment 19. The method of Embodiment 18, wherein the cells are selected from the group consisting of neurons, and stem cells.
Embodiment 20. The method of Embodiment 1, wherein the method does not comprise transecting a nearby healthy nerve or the repaired nerve.
Embodiment 21. The method of Embodiment 1, wherein the method enhances the survival of Schwann cells in the proximal nerve segment.
Embodiment 22. The method of Embodiment 1, wherein the proximal site is at least about 3 cm away from the site of injury.
Embodiment 23. The method of Embodiment 1, wherein the proximal site is less than least about 3 cm away from the site of injury.
Embodiment 24. The method of Embodiment 1, wherein the method comprises contacting multiple proximal nerve segments with one or more stretch-grown TENG.
Embodiment 25. The method of Embodiment 1, wherein the TENG facilitates axon growth and Schwann cell infiltration, thereby maintaining the pro-regenerative capacity of the proximal nerve segment.
Embodiment 26. The method of Embodiment 1, wherein the method further comprises transplanting a stretch-grown tissue engineered nerve graft (TENG) into a distal site in the distal nerve segment.
Embodiment 27. The method of Embodiment 1, wherein the TENG is a forced aggregation TENG.
Embodiment 28. The method of Embodiment 1, wherein the TENG is transplanted into a proximal site in a peripheral nerve, a cranial nerve or a spinal nerve of a subject.
Embodiment 29. A method for maintaining the health of motor neurons in the spinal cord and the pro-regenerative capacity of a proximal nerve segment subsequent to a nerve injury by transplanting one or more neurons into a proximal site in the proximal nerve segment, wherein the one or more neurons facilitate regeneration and functional following nerve repair.
Embodiment 30. The method of Embodiment 29, wherein the nerve injury comprises an injury to a peripheral nerve, a cranial nerve or a spinal cord nerve of the subject.
Embodiment 31. The method of Embodiment 29, wherein one or more neurons are transplanted into a peripheral nerve, a cranial nerve or a spinal cord nerve of the subject.
Embodiment 32. The method of Embodiment 29, wherein one or more neurons are injected into a proximal nerve stump of the subject.
Embodiment 33. The method of Embodiment 29, wherein one or more neurons are transplanted in a delivery device that is secured to the proximal nerve stump.
Embodiment 34. The method of Embodiment 33, wherein one or more neurons are encapsulated in extracellular matrix sheath and transferred into the delivery device.
Embodiment 35. The method of Embodiment 33, wherein one or more neurons are stretch-grown in culture, encapsulated in extracellular matrix, and transferred into the delivery device.
Embodiment 36. The method of Embodiment 33, wherein one or more neurons are pre-encapsulated in extracellular matrix, grown in culture, and then transferred into the delivery device.
Embodiment 37. The method of Embodiment 29, wherein one or more neurons are comprise glutamatergic, GABAergic, sensory neurons, motor neurons or combinations thereof.
Embodiment 38. The method of Embodiment 29, wherein one or more neurons are accompanied by Schwann cells, macrophages, fibroblasts, mesenchymal stem cells or myocytes.
Embodiment 39. The method of Embodiment 29, wherein one or more neurons is derived from neuronal progenitor cells.
Embodiment 40. The method of Embodiment 29, wherein one or more neurons is derived from stem cells.
Embodiment 41. The method of Embodiment 29, wherein one or more neurons is derived from embryonic cells.
Embodiment 42. The method of Embodiment 29, wherein the one or more neurons are implanted along or within an extracellular matrix core and are formed via forced cell aggregation.
Embodiment 43. The method of Embodiment 29, wherein one or more neurons, wherein one or more neurons are transduced to overexpress neurotrophic factors, including, but not limited to GDNF, BDNF, VEGF, or NGF.
Embodiment 44. The method of Embodiment 38, wherein one or more neurons are supplemented with cells transduced to overexpress neurotrophic factors, including, but not limited to GDNF, BDNF, VEGF, or NGF.
Embodiment 45. The method of Embodiment 29, wherein the nerve injury comprises the loss of a segment of nerve.
Embodiment 46. The method of Embodiment 29, wherein the nerve injury comprises a nerve lesion of less than about 1 cm in length.
Embodiment 47. The method of Embodiment 29, wherein the nerve injury comprises a nerve lesion of at least 1 cm in length.
Embodiment 48. The method of Embodiment 29, wherein the nerve injury comprises a nerve lesion of at least about 3 cm in length.
Embodiment 49. The method of Embodiment 29, wherein the nerve injury comprises a nerve lesion of at least about 5 cm in length.
Embodiment 50. The method of Embodiment 29, wherein the nerve injury comprises multiple nerve lesions.
Embodiment 51. The method of Embodiment 29, wherein the pro-regenerative capacity of the proximal nerve segment is maintained for at least about 16 weeks.
Embodiment 52. The method of Embodiment 29, wherein the pro-regenerative capacity of the proximal nerve segment is maintained until at least such time as proximal nerve axons regenerate across the nerve injury.
Embodiment 53. The method of Embodiment 29, wherein pro-regenerative capacity of the proximal nerve segment is maintained until at least such time as proximal nerve axons reinnervate distal targets
Embodiment 54. The method of Embodiment 29, wherein the method further comprises a primary procedure for nerve repair that results in a greater degree of functional recovery following repair of PNI, as compared to the degree of functional recovery that occurs following the primary procedure alone.
Embodiment 55. The method of Embodiment 29, wherein the method further comprises a primary procedure to preserve the regenerative capacity of the proximal nerve segment followed by a delayed nerve repair at a later time point that results in a greater degree of functional recovery following repair of PNI, as compared to the degree of functional recovery that occurs following the delayed repair alone.
Embodiment 56. The method of Embodiment 29, wherein the method is conducted in the absence of any other nerve repair.
Embodiment 57. The method of Embodiment 29, wherein the nerve injury occurs as a result of a trauma.
Embodiment 58. The method of Embodiment 29, wherein the nerve injury occurs as a result of a surgical procedure.
Embodiment 59. The method of Embodiment 29, wherein the nerve injury occurs as a result of patient positioning during surgery.
Embodiment 60. The method of Embodiment 29, wherein the nerve injury occurs as a result of a compression injury or a crush injury.
Embodiment 61. The method of Embodiment 29, wherein the injury occurs as a result of a disease or condition relating to a loss of motor or sensory nerve function.
Embodiment 62. The method of Embodiment 29, wherein the injury occurs as a result of a congenital anomaly.
Embodiment 63. The method of Embodiment 29, wherein the injury occurs as a result of an amputation.
Embodiment 64. The method of Embodiment 29, wherein the injury occurs as a result of complete or partial removal of an organ, tumor or tissue.
Embodiment 65. The method of Embodiment 29, wherein the injury occurs as a result of a metabolic/endocrine complication, inflammatory disease, autoimmune disease, vitamin deficiency, infectious disease, toxin, exposure to organic metal or heavy metal, or administration of a medication or drug.
Embodiment 66. The method of Embodiment 29, further comprising providing a neurotrophic factor, culture supernatant, or cells to the distal nerve segment.
Embodiment 67. The method of Embodiment 29, wherein the method does not comprise transecting a nearby healthy nerve or the repaired nerve.
Embodiment 68. The method of Embodiment 29, wherein the method does not comprise transecting a nearby healthy nerve or the repaired nerve.
Embodiment 69. The method of Embodiment 29, wherein the distal site is less than least about 3 cm away from the site of injury.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for maintaining the health of motor neurons in the spinal cord and the pro-regenerative capacity of a proximal nerve segment subsequent to a nerve injury in a subject in need thereof, the method comprising transplanting a stretch-grown tissue engineered nerve graft (TENG) into a proximal site contacting the proximal nerve segment.

2-3. (canceled)

4. The method of claim 1, wherein the nerve injury comprises an injury to a peripheral or cranial nerve of a subject.

5. The method of claim 1, wherein the nerve injury comprises an injury to the spinal cord of a subject.

6. The method of claim 1, wherein the nerve injury comprises the loss of a segment of nerve.

7. (canceled)

8. The method of claim 1, wherein the nerve injury comprises a nerve lesion of at least about 1 cm in length.

9-10. (canceled)

11. The method of claim 1, wherein the nerve injury comprises multiple nerve lesions.

12. (canceled)

13. The method of claim 1, wherein the pro-regenerative capacity of the proximal nerve segment is maintained until at least such time as proximal nerve axons regenerate across the nerve injury.

14. The method of claim 1, wherein pro-regenerative capacity of the proximal nerve segment is maintained until at least such time as proximal nerve axons reinnervate distal targets.

15. The method of claim 1, wherein the method further comprises a primary procedure for nerve repair.

16-17. (canceled)

18. The method of claim 1, further comprising providing a neurotrophic factor, culture supernatant, or cells to the proximal nerve segment.

19. The method of claim 18, wherein the cells are selected from the group consisting of neurons, and stem cells.

20-21. (canceled)

22. The method of claim 1, wherein the proximal site is at least about 3 cm away from the site of injury.

23. The method of claim 1, wherein the proximal site is less than least about 3 cm away from the site of injury.

24. The method of claim 1, wherein the method comprises contacting multiple proximal nerve segments with one or more stretch-grown TENG.

25. (canceled)

26. The method of claim 1, wherein the method further comprises transplanting a stretch-grown tissue engineered nerve graft (TENG) into a distal site in the distal nerve segment.

27. The method of claim 1, wherein the TENG is a forced aggregation TENG.

28. The method of claim 1, wherein the TENG is transplanted into a proximal site in a peripheral nerve, a cranial nerve or a spinal nerve of a subject.

29. A method for maintaining the health of motor neurons in the spinal cord and the pro-regenerative capacity of a proximal nerve segment subsequent to a nerve injury by transplanting one or more neurons into a proximal site in the proximal nerve segment, wherein the one or more neurons facilitate regeneration and functional following nerve repair.

30. The method of claim 29, wherein the nerve injury comprises an injury to a peripheral nerve, a cranial nerve or a spinal cord nerve of the subject.

31. The method of claim 29, wherein one or more neurons are transplanted into a peripheral nerve, a cranial nerve or a spinal cord nerve of the subject.

32. The method of claim 29, wherein one or more neurons are injected into a proximal nerve stump of the subject.

33. The method of claim 29, wherein one or more neurons are transplanted in a delivery device that is secured to the proximal nerve stump.

34. The method of claim 33, wherein one or more neurons are encapsulated in extracellular matrix sheath and transferred into the delivery device.

35. The method of claim 33, wherein one or more neurons are stretch-grown in culture, encapsulated in extracellular matrix, and transferred into the delivery device.

36. The method of claim 33, wherein one or more neurons are pre-encapsulated in extracellular matrix, grown in culture, and then transferred into the delivery device.

37. The method of claim 29, wherein one or more neurons are comprise glutamatergic, GABAergic, sensory neurons, motor neurons or combinations thereof.

38. The method of claim 29, wherein one or more neurons are accompanied by Schwann cells, macrophages, fibroblasts, mesenchymal stem cells or myocytes.

39. The method of claim 29, wherein one or more neurons is derived from neuronal progenitor cells.

40. The method of claim 29, wherein one or more neurons is derived from stem cells.

41. The method of claim 29, wherein one or more neurons is derived from embryonic cells.

42. The method of claim 29, wherein the one or more neurons are implanted along or within an extracellular matrix core and are formed via forced cell aggregation.

43-44. (canceled)

45. The method of claim 29, wherein the nerve injury comprises the loss of a segment of nerve.

46. (canceled)

47. The method of claim 29, wherein the nerve injury comprises a nerve lesion of at least 1 cm in length.

48-49. (canceled)

50. The method of claim 29, wherein the nerve injury comprises multiple nerve lesions.

51. (canceled)

52. The method of claim 29, wherein the pro-regenerative capacity of the proximal nerve segment is maintained until at least such time as proximal nerve axons regenerate across the nerve injury.

53. The method of claim 29, wherein pro-regenerative capacity of the proximal nerve segment is maintained until at least such time as proximal nerve axons reinnervate distal targets

54. The method of claim 29, wherein the method further comprises a primary procedure for nerve repair that results in a greater degree of functional recovery following repair of PNI, as compared to the degree of functional recovery that occurs following the primary procedure alone.

55. The method of claim 29, wherein the method further comprises a primary procedure to preserve the regenerative capacity of the proximal nerve segment followed by a delayed nerve repair at a later time point that results in a greater degree of functional recovery following repair of PNI, as compared to the degree of functional recovery that occurs following the delayed repair alone.

56-65. (canceled)

66. The method of claim 29, further comprising providing a neurotrophic factor, culture supernatant, or cells to the distal nerve segment.

67. The method of claim 29, wherein the method does not comprise transecting a nearby healthy nerve or the repaired nerve.

68. The method of claim 29, wherein the method does not comprise transecting a nearby healthy nerve or the repaired nerve.

69. The method of claim 29, wherein the distal site is less than least about 3 cm away from the site of injury.
